(19) United States
(12) Patent Application Publication
(10) Pub. No.: US 2011/0243866 A1
(43) Pub. Date: Oct. 6, 2011

(54) COSMETIC COMPOSITION FOR MAKE-UP AND PROCESS FOR MAKING SAME

(75) Inventor: Piero ORIGO, Mozzanica (IT)

(73) Assignee: ART COSMETICS S.R.L., Mozzanica (IT)

(21) Appl. No.: 13/031,835

(22) Filed: Feb. 22, 2011

(30) Foreign Application Priority Data
Feb. 23, 2010 (IT) MI2010A000286

(51) Int. Cl. A61K 8/92; A61Q 1/02

(52) U.S. Cl. 424/63

(57) ABSTRACT

A process for the preparation of a make-up cosmetic comprising: a) dryly preparing a powder mixture consisting of talc, mica, silica, colorants, pearls and binders, wherein colorants are present in percentages from 1 to 99%, pearls are present in percentages from 1 to 99%, and the binder are present in percentages from 1 to 99%; b) sieving the mixture obtained; c) wetting the mixture obtained at step b) with one or more oil-based compounds selected from the group consisting of: a mineral or vegetable oil, a fatty alcohol, an ester, a silicone oil, a triglyceride and a wax, in percentages ranging from 1% to 50%; d) adding the mixture obtained in step c) to an aqueous solution, suspension, dispersion or emulsion in percentages from 1 to 99%; e) depositing the mixture obtained in step d), manually, mechanically or with a press, on or in a support; and f) baking the mixture obtained in step e) in an oven at a temperature ranging from 5° C. to 150° C. and under a pressure ranging from 10 mm of mercury to 1520 mm of mercury.

The present invention relates to a cosmetic composition for make-up, and the process for its preparation.

### STATE OF THE ART

Cosmetic compositions in powder form, and in particular cosmetic compositions formulated for colouring the face, body and hair, commonly known as “make-up”, are made in different forms, such as: loose powder, compact powder, baked powder, cast powder and extruded powder. Examples of said compositions are described in patents EP 890356, EP 1053741, U.S. Pat. No. 12/358,562 and U.S. Pat. No. 12/068,053.

From the technical standpoint, all cosmetic compositions in powder form converge towards common objectives, which can be summarised as pleasant feel, easy to take up, excellent application to the skin, excellent colour, long duration, the possibility of using high percentages of raw materials known in cosmetics jargon as “pearls”, and using the product under dry or wet conditions, high resistance to mechanical stresses and transport, and maintenance of good stability to thermal and environmental variations.

The compositions currently present on the market only partly achieve the said objectives; above all, no formulation achieves them all simultaneously. To cite just a few examples, loose powders have low adherence to the skin and a low colour transfer capacity; compact powders have a rather unpleasant feel and a hardness which, in many cases, makes them difficult to take up, and often suffer from two problems known in cosmetics jargon as dusting or shine; baked powders are often too fragile during transport and handling; cast powders tend to soften as the temperature increases; extruded powders may be rubbery, and there are limitations on the maximum number of pearls that they can contain.

### DESCRIPTION OF THE INVENTION

It has been found that certain raw materials, when mixed in suitable ratios and processed by a suitable technique, allow make-up cosmetics with the following characteristics to be manufactured in powder form:
a feel that can be varied as required; for example, the same product can be given a rough or velvety feel;
take-up is excellent under all conditions of use; for example, the same product can be made in different varieties which can be taken up with the fingers or with a wide variety of applicators, under dry or wet conditions;
the capacity of transfer of the colour to the skin can be varied as required;
the degree of adherence to the skin after application is excellent;
colouring raw materials, known in cosmetics jargon as “pearls”, can be included, with no concentration limit;
high mechanical strength if dropped and high mechanical resistance to transport conditions;
inertia towards variations in climatic conditions (heat, cold, humidity).

The present invention therefore relates to a process for the manufacture of make-up cosmetics in powder form having the characteristics stated above, and cosmetics obtained by said process, in particular for use on the eyes, face, body and hair.

Said process comprises the following steps:
a) preparation of a mixture of powders with components such as talc, mica, silica, colorants, pearls and binders;
b) sieving of the mixture obtained;
c) wetting of the mixture with one or more oil-based compounds;
d) addition of an aqueous phase to create a stiff mixture;
e) deposit of the mixture on or in a support;
f) baking of the mixture in a oven under variable temperature, time and humidity conditions.

At step a), the various components are mixed dry in a grinder. The components used to prepare the step a) mixture will be present in percentages ranging between 1 and 99%. According to a preferred aspect of the invention, said components will be present in the following percentages by weight:
colorants between 5 and 30%;
pearls between 30 and 60%;
binders between 1 and 10%.

According to the invention, the colorants are selected from the group of synthetic organic compounds and inorganic compounds.

According to the invention the pearls are mixtures that reflect ultraviolet and/or visible light which contain mica and titanium dioxide in varying ratios, and variable percentages of various colorants, iron oxides, silica and tin oxide.

According to the invention the binders are selected from mixtures of oils, including liquid paraffin, triglycerides, long-chain alcohols and esters, silicone oils and/or gels obtained by polymerisation and copolymerisation of unsaturated aliphatic long-chain monomers.

At wetting step c) the oil-based compound can consist of a mineral or vegetable oil, a fatty alcohol, an ester, a silicone oil, a triglyceride or a wax, used individually or mixed in percentages ranging between 1 and 99%.

More particularly, the oil-based compounds used at wetting step c) are as follows:
saturated or unsaturated fatty alcohol
saturated or unsaturated long-chain ester
silicone oil
mineral oil
vegetable oil
triglycerides.

At step d) the aqueous phase can contain emulsifiers, binders, thickeners, active ingredients, preservatives, or a mixture thereof in addition to water. More particularly, the components used to prepare the aqueous solution of step d) are as follows:
anionic, cationic, non-ionic and amphoteric emulsifiers
xanthan gum
carboxymethylcellulose and hydroxymethylcellulose
acrylic and methacrylic acid polymers
glycerol
polysaccharides
preservatives
water.

At step f) the mixture is baked in an oven at a temperature ranging between 5° C. and 150° C., preferably at 60° C., and at a pressure ranging between 10 mmHg and 1520 mmHg, preferably 760 mmHg.

The examples given below illustrate the present invention more clearly.

### EXAMPLES

The following examples are included to describe the invention in greater detail, without limitation.

### Example 1

### Step (a)

| Talc | q. s. for 100% |
|---|---|
| Mica | 5% |
| Yellow iron oxide | 3% |
| Ultramarine | 4% |
| Pearls | 35% |
| Preservatives | 0.2% |
| Liquid paraffin | 1.5% |
| Zinc stearate | 0.5% |

Mix all the components of step (a) in a fan grinder for 10 minutes at 3600 rpm.

Sieve the product obtained from processing step (a) with a 60-mesh sieve.

### Step (c)

| | |
|---|---|
| Product obtained from processing step (b) | 80% |
| Silicone | 10% |
| Cetyl alcohol | 5% |
| Isopropyl myristate | 5% |

Create a mixture from the components of step (c)

### Step (d)

| | |
|---|---|
| Product obtained from processing step (c) | 75% |
| Water | 17.8% |
| Xanthan gum | 2% |
| Glycerol | 5% |
| Preservatives | 0.2% |

Create a mixture from the components of step (d)

### Step (e)

Deposit the product obtained from processing step (d) in portions suitable for the support selected, and compact it with a press.

### Step (f)

Bake the product obtained from processing step (e) in an oven at the temperature of 60° C. and the pressure of 760 mmHg for eight hours.

### Example 2

### Step (a)

| Mica | q.s. for 100% |
|---|---|
| Yellow iron oxide | 3% |
| Black iron oxide | 1% |
| Chromium oxide | 1% |
| Manganese violet | 4% |
| Pearls | 35% |
| Preservatives | 0.2% |
| Octyldodecanol | 3% |
| Zinc stearate | 1% |

Mix all the components of step a and grind them in a pin mill.

### Step (b)

Sieve the product obtained from processing step (a) with a 90-mesh sieve

### Step (c)

| | |
|---|---|
| Product obtained from processing step (b) | 80% |
| Silicone | 10% |
| Stearyl alcohol | 5% |
| Caprylic/capric triglyceride | 4% |
| Zinc stearate | 1% |

Create a mixture with the components of step c

### Step (d)

| | |
|---|---|
| Product obtained from processing step (c) | 75% |
| Water | 21.3% |
| Polyacrylate | 3% |
| Carboxymethylcellulose | 0.5% |
| Preservatives | 0.2% |

Create a mixture with the components of step d

### Step (e)

Deposit the product obtained from processing Step (d) in quantities suitable to fill the support selected, and compact it with a press.

### Step (f)

Bake the product obtained by processing step (e) in an oven at the temperature of 70° C. and the pressure of 760 mmHg for two hours.

1. A process for the preparation of a make-up cosmetic comprising:
a) dryly preparing a powder mixture consisting of talc, mica, silica, colorants, pearls and binders, wherein colorants are present in percentages from 1 to 99%, pearls are present in percentages from 1 to 99%, and the binder are present in percentages from 1 to 99%
b) sieving the mixture obtained;
c) wetting the mixture obtained at step b) with one or more oil-based compounds selected from the group consisting of: a mineral or vegetable oil, a fatty alcohol, an ester, a silicone oil, a triglyceride and a wax, in percentages ranging from 1% to 50%;
d) adding the mixture obtained in step c) to an aqueous solution, suspension, dispersion or emulsion in percentages from 1 to 99%;
e) depositing the mixture obtained in step d), manually, mechanically or with a press, on or in a support; and
f) baking the mixture obtained in step e) in an oven at a temperature ranging from 5° C. to 150° C. and under a pressure ranging from 10 mm of mercury to 1520 mm of mercury.

2. The process of claim 1, wherein in step c) the mixture obtained in step b) is wetted with a mixture of mineral or vegetable oil, a fatty alcohol, an ester, a silicone oil, a triglyceride or a wax, in the percentage of 20%.

3. The process of claim 1, wherein the mixture obtained at step e) is baked in an oven at the temperature of 60° C. and under a pressure of 760 mm of mercury.

4-7. (canceled)

8. The process of claim 1, wherein in step a) said colorants are present in percentages from 5 to 30%, said pearls are present in an amount from 30 to 60%, and said binder is present in an amount from 1 to 10%.

9. The process of claim 1, wherein in step d) said aqueous solution, suspension, dispersion or emulsion are in percentages from 10 to 30%.
